(19) United States
(12) Patent Application Publication
(10) Pub. No.: US 2026/0325015 A1
(43) Pub. Date: Oct. 22, 2026

(54) NONINVASIVE CONTINUOUS GLUCOSE MONITORING USING BANDPASS RAMAN SPECTROSCOPY

(71) Applicants: Massachusetts Institute of Technology, Cambridge, MA (US); Apollon Inc., Cheongju-si (KR)

(72) Inventors: Peter T. C. So, Boston, MA (US); Jeon Woong Kang, Melrose, MA (US); Arianna Bresci, Cambridge, MA (US); Youngkyu Kim, Seoul (KR)

(21) Appl. No.: 19/458,753

(22) Filed: Jan. 24, 2026

Related U.S. Application Data
(60) Provisional application No. 63/749,441, filed on Jan. 24, 2025.

(51) Int. Cl. A61B 5/145; A61B 5/1455

(52) U.S. Cl. CPC A61B 5/14532; A61B 5/1455

(57) ABSTRACT

A method for noninvasive continuous glucose monitoring (CGM) using bandpass Raman spectroscopy (BRS) includes: irradiating, by a light source, light to a subject; detecting, by a photodetector, the light that is reflected from the subject; obtaining intensities of peaks at one or more predetermined Raman bands in a Raman spectrum occurring due to Raman shift, wherein the one or more predetermined Raman bands include a glucose Raman peak and two sidebands; and obtaining at least one glucose metric based on the intensities of peaks at the one or more predetermined Raman bands. The glucose metric is an area metric and/or a slope metric. Based thereon, a glucose level associated with the subject is determined.

### CROSS-REFERENCE TO RELATED APPLICATION(S)

This application claims priority from Provisional Application No. 63/749,441 filed Jan. 24, 2025, which is incorporated herein by reference in its entirety.

### TECHNICAL FIELD

The present disclosure relates to blood glucose monitoring, and more particularly to noninvasive continuous glucose monitoring using bandpass Raman spectroscopy.

### RELATED ART

Diabetes has reached epidemic proportions worldwide, with projections indicating 592 million cases by 2035¹. Managing this chronic condition relies heavily on effective blood glucose monitoring, a cornerstone of diabetes treatment. Continuous glucose monitoring (CGM) systems have emerged as transformative tools not only for diabetes management but also for broader applications such as wellness tracking and athletic performance optimization². Despite their utility, traditional finger-pricking methods are impractical for continuous use, and current commercial CGMs, though effective, rely on invasive microneedles to measure interstitial fluid (IF) glucose levels, falling short of true noninvasiveness³.

Noninvasive approaches have sought to overcome these limitations through diverse technologies. Vibrational spectroscopy methods, including Raman⁴, near-infrared (NIR)⁵, and mid-infrared⁶ spectroscopy, directly target the molecular signatures of glucose. Techniques such as photothermal and photoacoustic spectroscopy leverage thermal or acoustic responses to glucose-specific absorption⁷. Optical coherence tomography⁸ and polarimetry⁹ measure refractive index changes and glucose chirality, respectively, while fluorescence-based methods use glucose-sensitive fluorophores¹⁰. Emerging modalities like terahertz spectroscopy¹¹, microwave¹² and radiofrequency¹³ sensing, ultrasound-assisted¹⁴ techniques, and bioimpedance spectroscopy¹⁵ exploit changes in tissue optical, electrical, or thermal properties. Indirect methods, including photoplethysmography¹⁶ and breath analysis¹⁷, infer glucose levels through secondary physiological effects or glucose metabolism by-products, while alternative fluid sampling (tears, saliva, sweat) provides minimally invasive options¹⁸.

Many of these methods increasingly rely on artificial intelligence (AI) to process complex and noisy signals, helping to retrieve glucose-specific metrics by mitigating background noise and artifacts¹⁹. However, heavy reliance on black-box AI technologies introduces challenges in explainability and generalizability, as these systems often require extensive training and may not adapt well to diverse patient populations or conditions. These limitations further emphasize the need for approaches that balance high sensitivity and specificity with transparency and robustness.

### SUMMARY

In order to address the challenges discussed above, an aspect of the present disclosure is to provide a bandpass Raman spectroscopy (BRS) method, which offers a compact and noninvasive solution for continuous glucose monitoring.

According to an aspect of the present disclosure, a method for noninvasive continuous glucose monitoring (CGM) using bandpass Raman spectroscopy (BRS) may include: irradiating, by a light source, light to a subject; detecting, by a photodetector, the light that is reflected from the subject; obtaining intensities of peaks at one or more predetermined Raman bands in a Raman spectrum occurring due to Raman shift, wherein the one or more predetermined Raman bands include a glucose Raman peak and two sidebands; obtaining at least one glucose metric based on the intensities of peaks at the one or more predetermined Raman bands; and determining a glucose level associated with the subject based on the at least one glucose metric.

The at least one glucose metric may correspond to an area of a triangle, which is defined by the intensities of the glucose Raman peak and the two sidebands, on a signal intensity—Raman shift plot.

The at least one glucose metric may correspond to a sum of: (i) absolute value of a first slope formed by a first line segment connecting the glucose Raman peak and one of the two sidebands on a signal intensity—Raman shift plot; and (ii) absolute value of a second slope formed by a second line segment connecting the glucose Raman peak and the other of the two sidebands on the signal intensity—Raman shift plot.

The following aspects may be included individually or in any combinations.

The light may include infrared (IR) light. The wavelength of the IR light may be about 830 nm.

The glucose Raman peak may correspond to a peak at about 1125 cm⁻¹, and the two sidebands may correspond to peaks at about 950 cm⁻¹ and about 1175 cm⁻¹.

The glucose Raman peak may be obtained using a filter having a central wavelength of about 915.5 nm, and the two sidebands may be obtained using filters having central wavelengths of about 901.3 nm and about 920.15 nm, respectively.

The method may further include normalizing raw signal of the intensities by dividing by correspondent reference signal, thereby obtaining a corrected Raman data, wherein the reference signal is obtained by routing the light from the light source to a photodetector without directing it to the subject.

The method may further include subtracting signals that represent a baseline glucose concentration from the corrected Raman data, thereby obtaining a corrected adjusted Raman data. By way of example, the baseline glucose concentration may represent the lowest glucose concentration measured. For example, the lowest glucose concentration may be 0 mg/dL.

The method may further include applying respective weights to each Raman band by optical bandwidth of corresponding bandpass filter, thereby obtaining a corrected weighted Raman data.

The method may further include subtracting signals that represent the lowest glucose concentration from the corrected weighted Raman data, thereby obtaining a corrected weighted adjusted Raman data.

The glucose level associated with the subject may be determined based on mapping the at least one glucose metric to a quadratic fitting of prior measurements of plurality of glucose concentrations.

An aspect of the present disclosure provides a system for noninvasive continuous glucose monitoring (CGM) of a human subject, comprising
a. A means of immobilizing an arm of the subject;
b. a source of infrared (IR) light configured to irradiate the arm of the subject;
c. a photodetector configured to measure the intensity of light reflected from the arm of the subject;
d. a means of collecting scattered light from the arm of the subject, comprising
   i. one or more bandpass filters;
   ii. an amplified photodiode for detecting scattered light;
   iii. a means of converting the detected scattered light to a glucose Raman signal.

In one aspect of the disclosed system, the IR light may include 830 nm near-infrared illumination.

In one aspect of the disclosed system, the source of the IR light may be a laser.

In one aspect of the disclosed system, the scattered light may be collimated using an achromatic doublet lens.

In one aspect of the disclosed system, the means of converting the scattered light to a glucose Raman signal may include a computer or a processor configured for data analysis and statistical validation.

### BRIEF DESCRIPTION OF THE DRAWINGS

A brief description of each drawing is provided to more sufficiently understand drawings used in the detailed description of the present disclosure.

FIG. 1A shows a schematic overview of a BRS-based CGM according to the present disclosure.

FIG. 1B schematically depicts the optical system for BRS according to an exemplary embodiment of the present disclosure.

FIG. 1C shows measured full-spectrum Raman signal of individual components and modelled tissue phantom signals.

FIG. 1D shows simulations of Raman spectra around the most prominent Raman peak of glucose (i.e., 1125 cm⁻¹) through 11 tissue phantoms glucose levels.

FIGS. 2A-2C show transmission curves of the ultra-narrowband BPFs impact on the recorded Raman signal.

FIG. 3 shows BRS glucose signal pre-processing schemes according to the present disclosure.

FIG. 4 shows metrics formulas according to the present disclosure and their typical behavior at different glucose levels.

FIGS. 5A-5D show metrics scaling proportionally with the glucose-specific Raman signal.

FIGS. 6A-6D illustrate BRS-based in-vivo intra-skin CGM on humans.

FIG. 7 shows step-by-step data analysis pipeline for clinical trial data of healthy humans.

It should be understood that the above-referenced drawings are not necessarily to scale, presenting a somewhat simplified representation of various preferred features illustrative of the basic principles of the disclosure. The specific design features of the present disclosure, including, for example, specific dimensions, orientations, locations, and shapes, will be determined in part by the particular intended application and use environment.

### DETAILED DESCRIPTION

Advantages and features of the present disclosure and a method of achieving the same will become apparent with reference to the accompanying drawings and exemplary embodiments described below in detail. However, the present disclosure is not limited to the exemplary embodiments described herein and may be embodied in variations and modifications. The exemplary embodiments are provided merely to allow one of ordinary skill in the art to understand the scope of the present disclosure, which will be defined by the scope of the claims. Accordingly, in some embodiments, well-known operations of a process, well-known structures, and well-known technologies will not be described in detail to avoid obscure understanding of the present disclosure. Throughout the specification, same reference numerals refer to same elements.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Reference numbers in superscripts herein refer to the corresponding literature listed in the Reference section which forms a part of this Specification. All references are incorporated by reference in their entireties.

Hereinabove, although the present disclosure is described by specific matters such as concrete components, and the like, the exemplary embodiments and the drawings are provided merely for assisting in the entire understanding of the present disclosure. Therefore, the present disclosure is not limited to the exemplary embodiments described herein. Various modifications and changes can be made by a person of ordinary skill in the art to which the present disclosure pertains. The spirit of the present disclosure should not be limited to the above-described exemplary embodiments, and the following claims as well as all technical spirits modified equally or equivalently to the claims should be interpreted to fall within the scope and spirit of the disclosure.

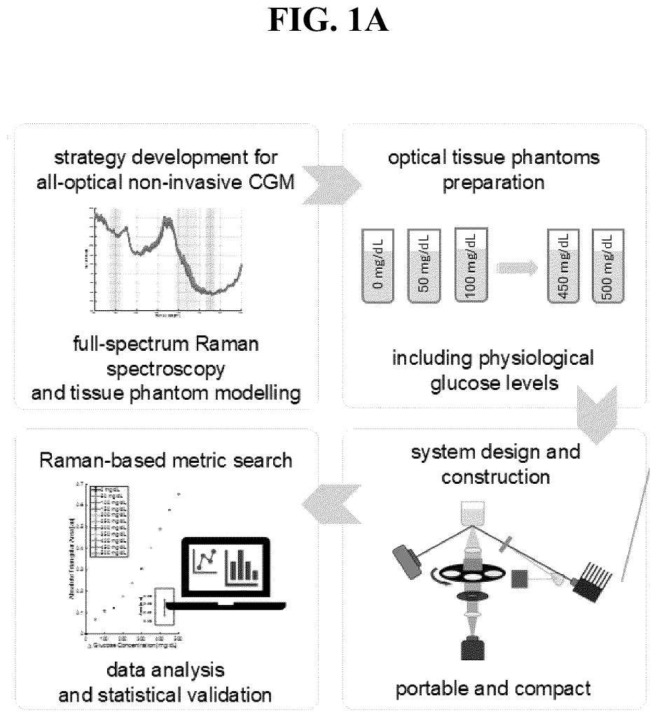

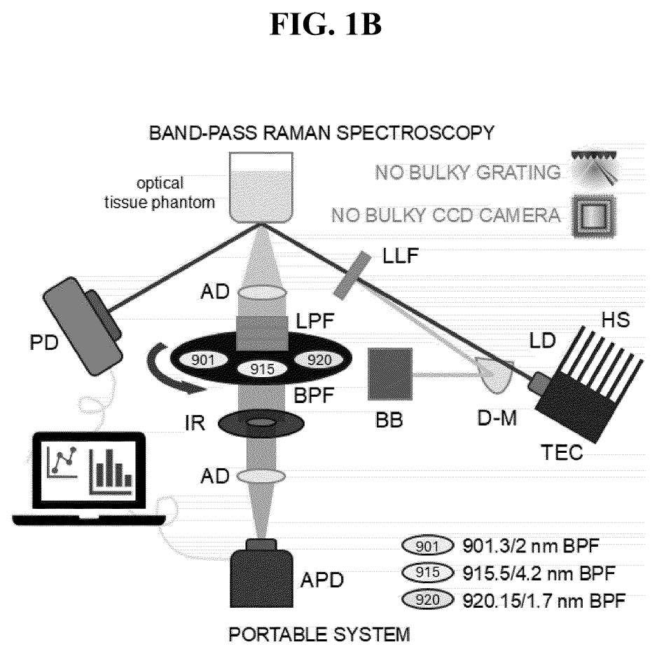

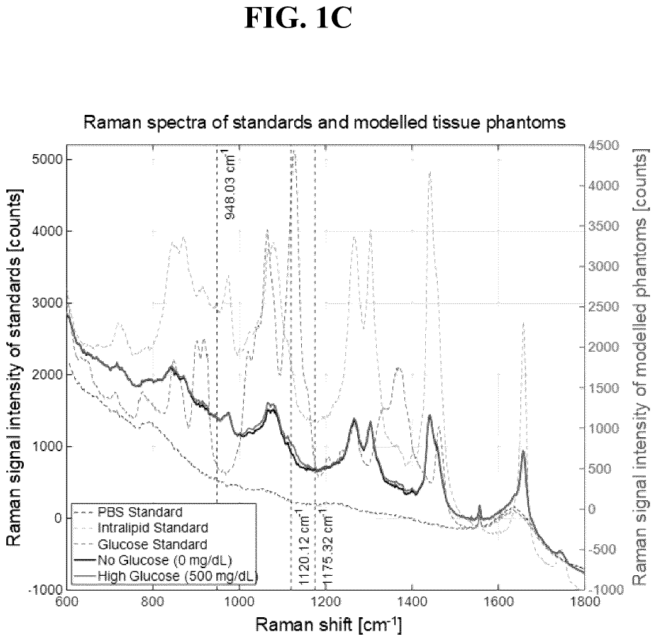

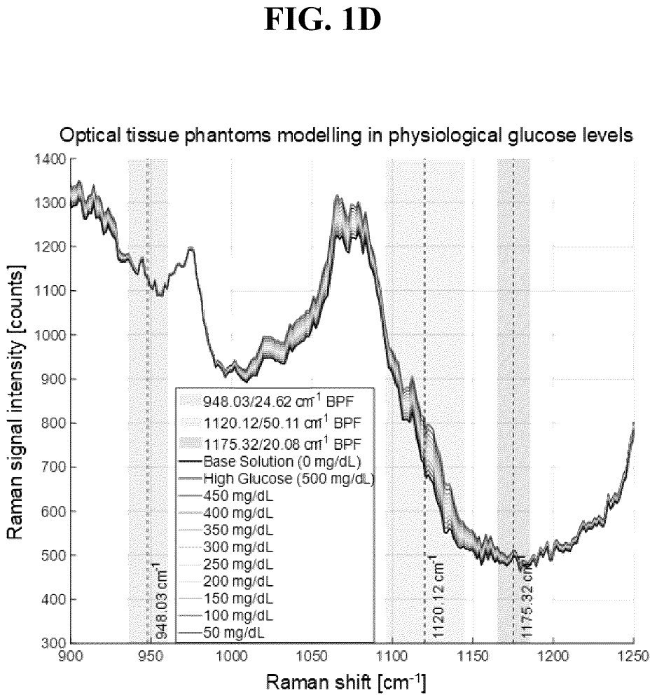

FIG. 1A shows a schematic overview of a BRS-based CGM according to the present disclosure. The expected signal is studied through full-spectrum Raman spectroscopy; optical tissue phantoms are produced to simulate the in-vivo IF; BRS is ideated to achieve practical and portable noninvasive CGM; a data processing pipeline is developed to extract glucose metric.

FIG. 1B schematically depicts the optical system for BRS according to an exemplary embodiment of the present disclosure. The BRS may include a heat sink (HS); a temperature control (TEC); a D-shaped mirror (D-M); a beam blocker (BB); a laser line filter (LLF); an achromatic doublet (AD); a longpass filter (LPF); a bandpass filter (BPF); an iris (IR); an amplified photodiode (APD); and a photodiode (PD).

FIG. 1C shows measured full-spectrum Raman signal of individual components and modelled tissue phantom signals: PBS (dashed blue line), 20% intralipid solution in water (dashed green line); 20% glucose solution in water (dashed red line); simulated no glucose tissue phantom (black line); and simulated high glucose tissue phantom (pink line).

FIG. 1D shows simulations of Raman spectra around the most prominent Raman peak of glucose (i.e., 1125 cm⁻¹) through 11 tissue phantoms glucose levels. Shaded areas indicate the chosen spectral bands for BRS according to the present disclosure.

### Full-Spectrum Raman Spectroscopy and Tissue Phantom Modelling

For a targeted approach for glucose detection, a full-spectrum dispersive Raman spectroscopy system was used to record the Raman spectra of individual components of tissue phantoms. The system was used to analyze a 20% glucose solution in water (Sigma-Aldrich, product code: 49163), a 20% intralipid emulsion (IL) (Sigma-Aldrich, product code: I141), and a phosphate-buffered saline solution (PBS) (Gibco, product code: 10010023). The spectral analysis focused on the 600-1800 cm⁻¹ region, known as the informative fingerprint region of the Raman spectrum. This range captures the most prominent glucose Raman peak at 1125 cm⁻¹, which is effective in monitoring glucose levels intra-skin. To simulate realistic tissue phantom full Raman spectra, a linear combination of the Raman spectra of the three components were applied as shown in Eq. 1:

I_{simulated}(v)=α·I_{glucose}(v)+β·I_{intralipid}(v)+γ·I_{PBS}(v) [Eq. 1]

where α, β, and γ represent the respective weight fractions of glucose, IL, and PBS. The weights of the three components were adjusted accordingly in the simulated tissue phantoms to reflect different glucose concentrations and produce their expected Raman spectra. Using this model, the Raman spectrum was simulated for a no-glucose tissue phantom (25% intralipid emulsion and 75% PBS) and a high-glucose tissue phantom with 500 mg/dL glucose (maintaining a constant 25% intralipid emulsion). Analysis of the simulated spectra confirmed that the most prominent glucose Raman peak at 1125 cm⁻¹ effectively distinguishes no-glucose and high-glucose conditions, with a clear spectral separation.

Further, Raman spectra were simulated for nine additional phantoms through physiological glucose concentrations ranging from 50 mg/dL to 500 mg/dL in 50 mg/dL increments. These simulations verified the direct relationship between Raman intensity and glucose concentration, consistent with the theoretical scaling of spontaneous Raman signals with scatterer concentrations in the focal volume. The parameters for optical tissue phantoms modelling are listed in Table 1.

TABLE 1

| Glucose Concentration | Alpha | Beta | Gamma |
|---|---|---|---|
| (mg/dL) | (Glucose) | (Intralipid) | (PBS) |
| 0 (no glucose) | 0 | 0.25 | 0.75 |
| 500 (high glucose) | 0.2 | 0.25 | 0.55 |
| 450 | 0.18 | 0.25 | 0.57 |
| 400 | 0.17777 | 0.25 | 0.57223 |
| 350 | 0.175 | 0.25 | 0.575 |
| 300 | 0.171433 | 0.25 | 0.578567 |
| 250 | 0.166667 | 0.25 | 0.583333 |
| 200 | 0.16 | 0.25 | 0.59 |
| 150 | 0.15 | 0.25 | 0.6 |
| 100 | 0.133333 | 0.25 | 0.616667 |
| 50 | 0.1 | 0.25 | 0.65 |

### Three Raman Spectral Band Representation

According to the present disclosure, instead of sampling standard full-spectrum Raman spectroscopy, three optimal Raman spectral regions may be sampled via bandpass Raman spectroscopy (BRS). More specifically, in addition to the glucose Raman peak at about 1125 cm⁻¹, two stable sidebands at about 950 cm⁻¹ and about 1175 cm⁻¹ may be selected to compensate for background variations and enable accurate retrieval of the glucose signal across varying concentrations with an intra-spectrum reference.

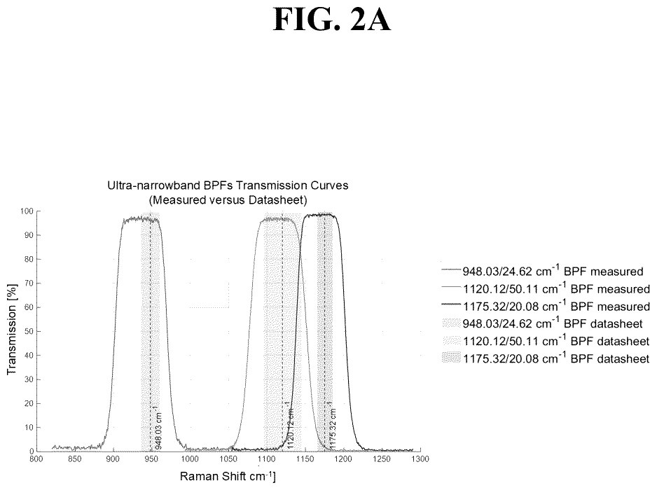

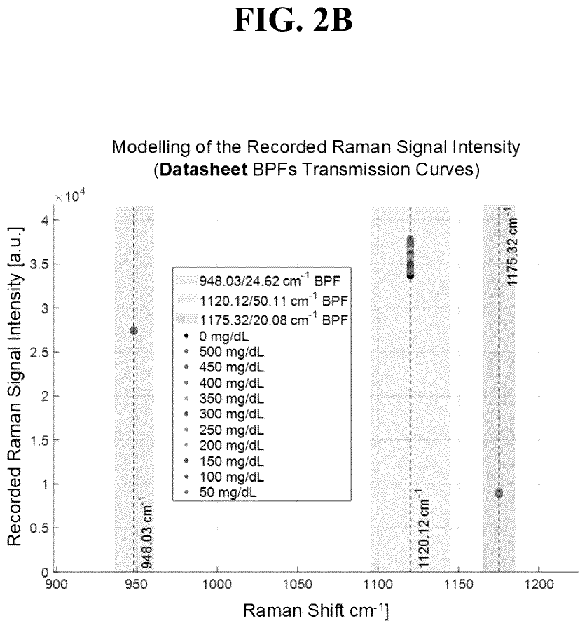

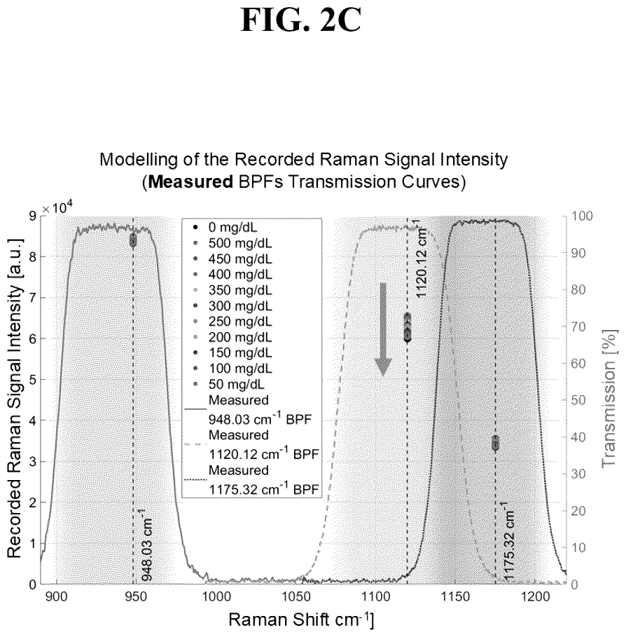

In some embodiments, customized filters (Alluxa) at 948.03/24.62 cm⁻¹ and 1175.32/20.08 cm⁻¹ may be used for the glucose peak sidebands, and 1120.12/50.11 cm⁻¹ for the glucose peak. These customized filters correspond to central wavelengths/bandwidths of 901.3/2 nm for the 950 cm⁻¹ sideband, 920.15/1.7 nm for the 1175 cm⁻¹ sideband, and 915.5/4.2 nm for the glucose peak, given the system 830.35 nm continuous-wave NIR laser source. FIGS. 2A-2C show transmission curves of the ultra-narrowband BPFs impact on the recorded Raman signal.

FIG. 2A shows ultra-narrowband BPFs transmission curves measured and averaged over a 10 pieces batch (lines) versus datasheet specifications (shaded areas). The measured transmission curves appear as larger in bandwidth and show a slight shift in terms of central bandwidth. More in detail, the 948.03 cm⁻¹ BPF features a measured lower Raman Shift (at 50% max transmission) at 901.80 cm⁻¹ and an upper Raman Shift (at 50% max transmission) at 970.14 cm⁻¹, resulting in a 936/68.35 cm⁻¹ BPF; the 1120.12 cm⁻¹ BPF features a measured lower Raman Shift (at 50% max transmission) at 1076.52 cm⁻¹ and an upper Raman Shift (at 50% max transmission) at 1152.00 cm⁻¹, resulting in a 1114.26/75.49 cm⁻¹ BPF; the 1175.32 cm⁻¹ BPF features a measured lower Raman Shift (at 50% max transmission) at 1137.75 cm-1 and an upper Raman Shift (at 50% max transmission) at 1202.30 cm⁻¹, resulting in a 1170.03/64.55 cm⁻¹ BPF.

FIG. 2B shows when considering the datasheet BPFs transmission curves at the selected wavelengths (while still excluding any detector quantum efficiency effect and other instrument parameters), the shape of the BRS spectrum recorded appears as upright triangular. The recorded Raman signal intensity is simulated as an integration of the area below the curve of the simulated tissue phantoms full Raman spectrum in FIG. 1D.

FIG. 2C shows when considering the measured BPFs transmission curves, the shape of the BRS spectrum more closely resembles the inverted triangular shape of the experimental one in tissue phantoms, with the central peak significantly decreasing in relative intensity compared to the sidebands intensity.

Such strategic band selection, using appropriate filters, may enable BRS of IF glucose levels circumventing the need for a grating and a CCD camera as the photon energies of interest are precisely isolated via bandpass optical filtering and directly recorded through an amplified photodiode, as detailed below.

### Bandpass Raman Spectroscopy (BRS) System

The bandpass Raman spectroscopy (BRS) system according to the present disclosure can provide compact, efficient, and precise glucose quantification through optimized optics and detection components. In some embodiments, the system may use a collimated laser beam at 830.35 nm (IPS, model: I0830S50135B-GUTS) with a laser diode current of 275 mA and a thermoelectric cooler (TEC) temperature set to 25° C. A heat sink (HS) may be mounted onto the laser diode to ensure thermal dissipation and stabilize laser power during operation, which is especially crucial when the optical setup is fully enclosed in a portable box.

The laser beam may be focused onto the tissue surface at an incidence angle of about 60°, forming an elliptical beam approximately 0.5 mm×1 mm in size. The inclined illumination geometry may minimize the detection of reflected photons by excluding them from the detection path, which is oriented normally to the sample surface. To spectrally purify the laser output, a laser line filter (Semrock, model: LL01-830-12.5) may be included with a slight inclination of 1-2° to deflect back reflected photons, preventing them from reentering the laser diode. The deflected beam may be suppressed using a beam blocker (Thorlabs, model: LB1).

Raman photons, which carry vibrational information specific to glucose molecules, are typically overwhelmed by scattered photons that lack such specificity. To collect the photons, the scattered light may be collimated using an achromatic doublet lens with a 20-mm focal length (Thorlabs, model: AC127-025-B). To isolate Raman photons from unwanted background signals, three sequential longpass filters (Semrock, model: BLP01-830R) may be disposed at the start of the detection branch. These filters may be followed by a motorized filter wheel (Thorlabs, model: FW212C), which houses the above-described ultra-narrowband bandpass filters at central wavelengths of 901.3 nm, 915.5 nm, and 920.15 nm. The filter wheel may be configured to rotate sequentially, allowing for sequential BRS signal acquisition.

To further minimize stray light, a 5-mm iris may be placed downstream, cutting photons propagating at significant angles. The filtered Raman photons may then be focused onto an amplified femtowatt photoreceiver equipped with a silicon photodiode (Femto, model: FWPR-20-SI) using another identical achromatic doublet lens (Thorlabs, model: AC127-025-B). A secondary detection path may collect reflected photons, which provide reference information regarding fluctuations in laser power through each BRS acquisition and give insight into the changes in refractive properties of the sample. It may use a photomultiplier tube (Thorlabs, model: PDA40) through a neutral density filter (Thorlabs, model: NE40A-B). The reflected signal may serve as a vital calibration reference to ensure comparability of signals through each sequential three-band BRS measurement.

Both the APD and the PD detectors may be connected to a data acquisition (DAQ) board (National Instruments, model: USB-6211) operating at 100 kHz, and a custom-designed MATLAB application may be used for opto-mechanical control, signal acquisition, and data saving. The entire optical system may be mounted on breadboards and enclosed to shield stray light, resulting in a compact and portable configuration with dimensions of 31×27×21 cm. A sample holder equipped with a quartz window may be mounted on top of the device, allowing convenient access for tissue preparation and dilution tests.

### Signal Processing, Metric Search and Statistical Validation

Using the BRS system described above, Raman-based metrics that scale proportionally with glucose levels in the sample were derived. For each three-band BRS measurement of glucose, only about 40 seconds are required: 10 seconds of continuous data acquisition per Raman band, preceded by a 3-second waiting period to minimize noise resulting from the mechanical movement of the motorized filter wheel.

DAQ samples may be averaged every second (100,000 samples) to mitigate noise, producing raw data for the APD-detected Raman signal and reference data for the PD-detected signal.

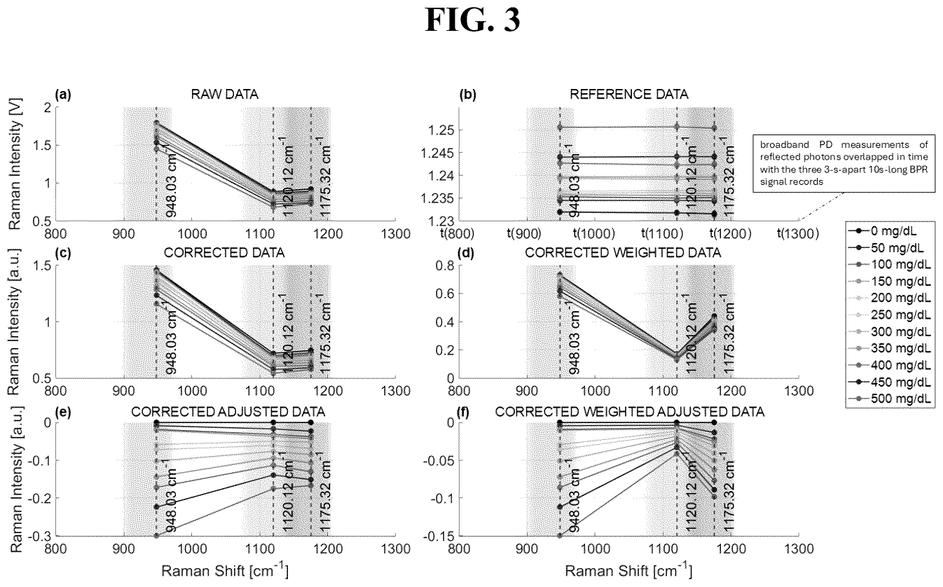

FIG. 3 shows BRS glucose signal pre-processing steps according to the present disclosure. Panel (a) of FIG. 3 depicts raw APD-detected Raman signals, 1-s-averaged 10-s-long measurements per Raman band. Panel (b) of FIG. 3 depicts raw PD-detected reference signals, 1-s-averaged 10-s-long measurements. It is worth noticing that the no bandpass optical filter precedes the reference PD in the system, hence all the broadband reflection is measured. However, shaded areas are reported to associate the measured reflected intensity to the temporal window of each Raman band sequentially recorded. Panel (c) of FIG. 3 depicts raw data corrected by the reference signal. Panel (d) of FIG. 3 depicts data corrected and weighted by the filter bandwidths. Panel (e) of FIG. 3 depicts data corrected and adjusted by the lowest concentration signal (i.e., 0 mg/dL in this example). Panel (f) of FIG. 3 depicts data corrected, weighted, and adjusted by the lowest concentration signal (i.e., 0 mg/dL in this example).

According to the present disclosure, to account for laser power fluctuations during the three-band BRS measurement, the raw data may be normalized by division of their correspondent reference signal, yielding corrected data (panel (c) of FIG. 3). Each band may then be weighted by the optical bandwidth of its corresponding bandpass filter in nanometers, producing the weighted data (panel (d) of FIG. 3). Differential glucose concentration may also be targeted, as opposed to absolute glucose levels: the lowest concentration signal (i.e., the 0 mg/dL tissue phantom) can be subtracted from the higher concentration signals, resulting in corrected adjusted data (panel (e) of FIG. 3) or corrected weighted adjusted data (panel (f) of FIG. 3).

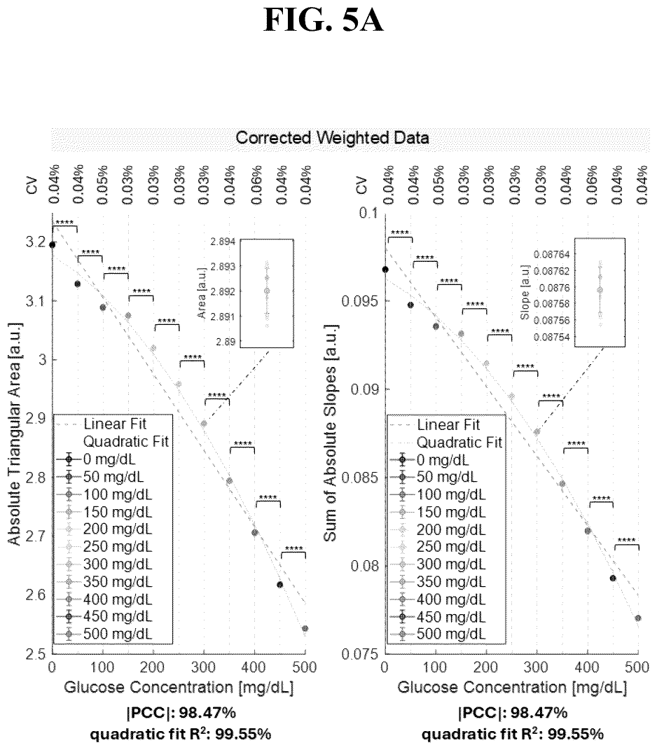

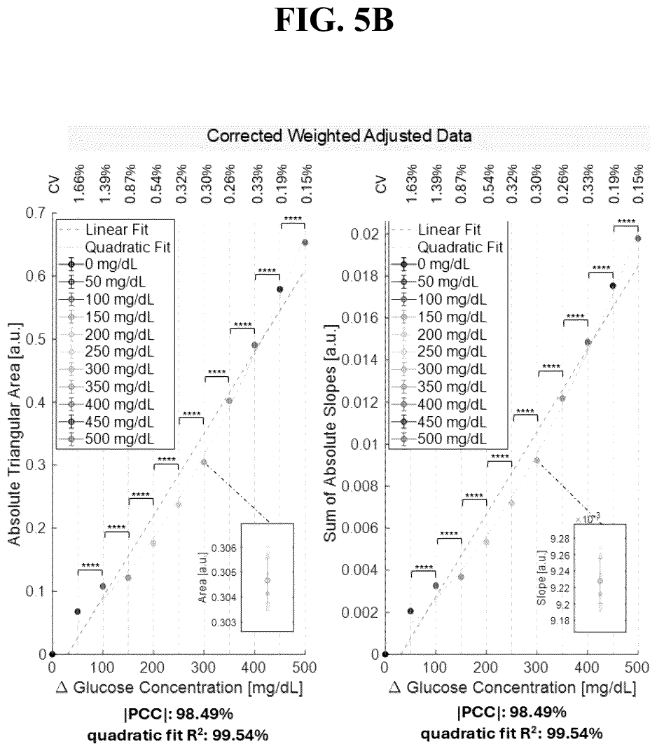

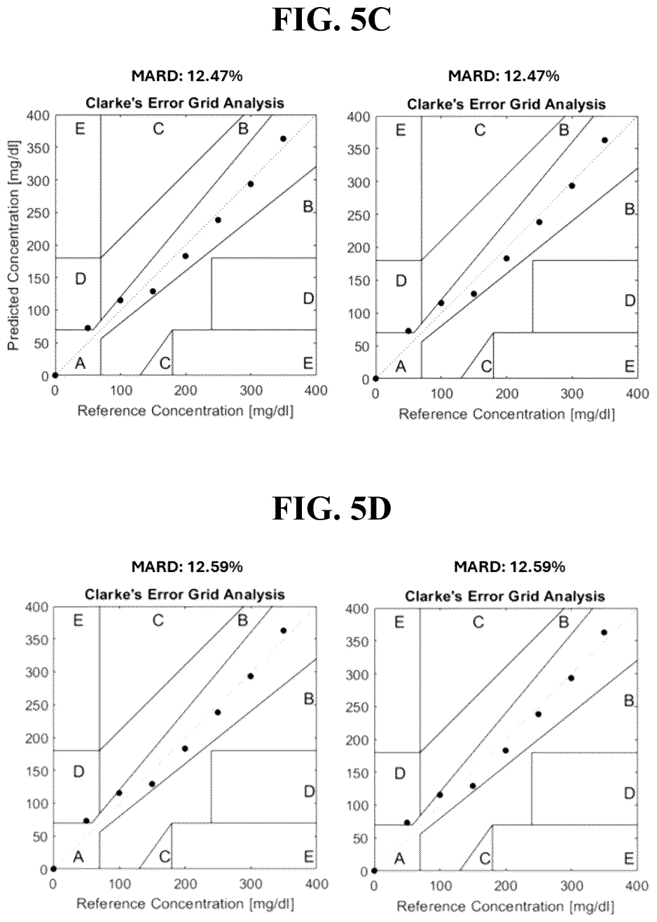

While processed datasets (panels (c)-(f) of FIG. 3) enhance differences between signals at varying glucose concentrations and simplify metric searches, raw Raman (panel (a) of FIG. 3) and reference (panel (b) of FIG. 3) signals provide valuable physical insights into the sample optical properties. The raw experimental data match the simulations of the expected Raman signal from the optical tissue phantoms (FIGS. 1D and 2A-2C). In particular, the Raman band corresponding to the 901.3-nm central wavelength features a clearly higher intensity compared to other bands, as higher scattering occurs in this spectral region according to the models (FIG. 1D). As for the reference data, it was observed that higher glucose levels lead to a higher reflected intensity, and vice versa. As glucose concentration increases within the voxel, the tissue phantom RI also rises due to the higher optical density introduced by glucose molecules. This increase in refractive index enhances the mismatch at the focal interface and, as described by Fresnel's equations, leads to increased light reflection (panel (b) of FIG. 3). Consequently, the photon flux available for Raman scattering diminishes, leading to a decrease in total Raman scattering intensity detected via the APD channel through all the three bands (panel (a) of FIG. 3 and also FIG. 5C). Despite the reduced overall photon count, the glucose signature remains detectable due to its unique spectral feature. In fact, by employing intra-spectrum references, such as the stable Raman sidebands of the glucose peak that the method of the present disclosure utilizes, the relative intensity of the glucose-specific peak can be successfully quantified, as described in the following.

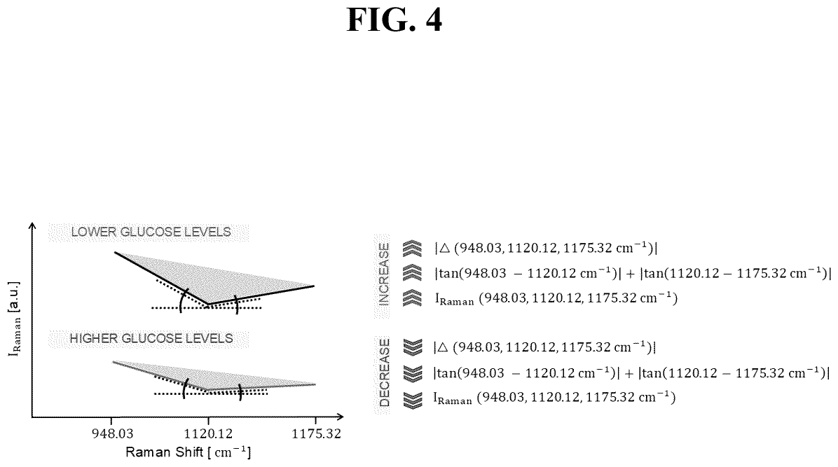

According to aspects of the present disclosure, referring to FIG. 4, metrics may be computed from the processed data and may include the absolute area encompassed by the signal at the three Raman bands, namely:

$$Area metric = \frac{1}{2} \vert ( 948.03 ( {I}_{1120.12} - {I}_{1175.32} ) + 1120.12 ( {I}_{1175.32} - {I}_{948.03} ) + 1175.32 ( {I}_{948.03} - {I}_{1120.12} ) ) \vert ,$$

and/or the sum of the two absolute slopes between the bands, namely:

$$Slope metric = \vert \frac{{I}_{1120.12} - {I}_{948.03}}{1120.12 - 948.03} \vert + \vert \frac{{I}_{1175.32} - {I}_{1120.12}}{1175.32 - 1120.12} \vert .$$

In other words, the area metric may correspond to an area of a triangle, which is defined by the intensity points of the glucose Raman peak (e.g., 1125 cm⁻¹ peak) and the two sideband peaks (e.g., 950 cm⁻¹ and 1175 cm⁻¹ peaks) on the signal intensity—Raman shift plot. In the present disclosure, the examples are presented using “a.u.” as the unit of signal intensity and “cm-1” as the unit of Raman shift. However, the present disclosure is not limited thereto. The glucose metrics may be derived based on other units, as long as the units are used in a consistent manner.

The slope metric may correspond to a sum of:
absolute value of the slope formed by a line segment connecting the glucose Raman peak (e.g., 1125 cm⁻¹ peak) and one of the sideband peaks (e.g., 950 cm⁻¹ peak) on the signal intensity—Raman shift plot; and
absolute value of the slope formed by a line segment connecting the glucose Raman peak (e.g., 1125 cm⁻¹ peak) and the other of the sideband peaks (e.g., 1175 cm⁻¹ peak) on the signal intensity—Raman shift plot.

In some embodiments, to mitigate noise, the 10-second averaged signals from the sidebands and the 1-second averaged signal from the Raman glucose band may be used for every 10-second band measurement. Metrics may be computed after weighting the data for the different filter bandwidths at the three Raman bands. This correction may effectively reduce the standard deviation of the metric values, more reliably unraveling their proportionality with respect to the glucose levels. This may result in a coefficient of variation (CV)<0.1% and CV<1.5% for metrics derived from corrected weighted data and corrected weighted adjusted data, respectively (the CV statistically measures the relative dispersion of data points around the mean). Naturally, CV values increase in adjusted data, in which the variation is affected by both the noise of the current measurement and the noise of the lowest glucose concentration measurement used for the adjustment.

The relationship between the Raman-based metrics and glucose levels was analyzed through the computation of the absolute Pearson correlation coefficient (PCC). FIGS. 5A-5D show metrics scaling proportionally with the glucose-specific Raman signal. FIG. 5A shows absolute area and sum of absolute slopes metrics calculated on Raman signals corrected by the reference laser power and weighted by the different filter bandwidths. FIG. 5B shows adjusted subtracting the signals at the lowest concentration measured (i.e., 0 mg/dL in this example). FIG. 5C shows statistically significant differences between adjacent glucose levels tested via Student T-testing, after assessing the normal distribution of data points collected via the Lilliefors (Kolmogorov-Smirnov) test. Significance is marked as: **** p-value<0.0001. The 0 mg/dL data cloud in the weighted adjusted dataset is not tested against other glucose levels for statistical difference as it nulls after adjustment with itself. Clarke error grid analysis of the quadratically calibrated Raman glucose versus the actual tissue phantom glucose concentration, for corrected weighted data. FIG. 5D shows corrected weighted adjusted data.

When non-adjusted data are used (FIG. 5A), the metrics scale with the absolute glucose concentration. Specifically, both the area and slope metrics decrease with higher glucose levels. This behavior occurs because, as glucose concentration increases, the Raman peak of glucose rises in intensity and moves closer to the intensities of the higher sidebands, resulting in a decrease in the overall encompassed area and absolute slopes values. Conversely, for lower glucose concentrations, the metrics exhibit higher values, reflecting the greater contrast between the Raman glucose peak and the sidebands (FIG. 5C). Clearly, this metrics behavior is typical when the measured BRS spectrum forms an inverse triangular shape, with the glucose peak as the lower intensity vertex (panel (a) of FIG. 3). In fact, the observed shape of the bandpass Raman spectrum, whether slightly positive or slightly negative triangular, is entirely acceptable and does not impact the reliability of the metrics. This variation arises from a combination of instrumental factors, including the bandwidth and transmission efficiency of the bandpass filters and the quantum efficiency, as well as the scattering properties dictated by the tissue phantom formula. However, the metrics remain robust to these variations, as their proportional relationship to glucose levels is preserved. If such tissue phantom or system changes result in a positive triangle-shaped BRS spectrum, the same metrics can be applied but will scale directly with absolute glucose levels instead.

On the other hand, when targeting differential glucose concentrations after adjusting the signals by subtracting the lowest concentration ones (FIG. 5B), the metrics exhibit direct proportionality with glucose levels. This is due to the differential nature of the processed data: higher glucose concentrations produce a larger absolute area and sum of slopes difference compared to the lowest measured concentration used as the baseline for adjustment (panel (f) of FIG. 3).

The PCC between the absolute area and sum of absolute slopes metrics in corrected weighted data was 98.47% (FIG. 5A), while the PCC in corrected weighted adjusted data was 98.49% (FIG. 5B). Such high values indicate a strong linear relationship between the two metrics and glucose levels, suggesting their consistency and reliability in tracking glucose. To gain deeper insight into how the metrics scale with glucose concentration, their behavior was analyzed using linear and quadratic fits. For both the corrected weighted and corrected weighted adjusted data, the linear fit R² value is 97%, while the quadratic fit R² value is notably higher at 99.5% (FIGS. 5A and 5B), for both metrics. This indicates that the metrics follow a predominantly quadratic relationship with glucose levels in the voxel. The superior performance of the quadratic fit highlights the suitability of a quadratic calibration strategy, which offers improved accuracy and precision for the application of the system in glucose quantification.

Building on these findings, a quadratic calibration of the Raman-based metrics was implemented to evaluate their performance in predicting glucose levels. The results, presented in FIG. 5C, demonstrate that the mean absolute relative difference (MARD) between the predicted and actual glucose concentrations is 12.47% when using corrected adjusted data, and 12.59% when using corrected weighted adjusted data (FIG. 5D). These MARD values represent a high level of accuracy, as a MARD below 15% is often considered acceptable for CGM systems at a clinical level.

To further validate the robustness of the approach on tissue phantoms, a Clarke error grid analysis was performed, which is a method commonly applied for glucose levels up to 400 mg/dL. The Clarke error grids for both metrics and data preprocessing strategies exhibit comparable results, with a predominance of observations falling into the clinically acceptable A zone, where deviations are considered non-significant for therapeutic decisions. Only one out of seven observations lies on the border within the D zone, which represents regions of potentially dangerous clinical errors. This proves the reliability and robustness of the method for systematic data analysis according to the present disclosure.

### Clinical Intra-Skin Application to Humans

To evaluate the feasibility of the Raman-based CGM system in a clinical setting, a preliminary trial was conducted on a healthy participant with a Fitzpatrick skin type II. The participant was monitored over approximately four hours, and measurements were taken every five minutes using the Raman-based portable system on the non-dominant forearm. For comparison, two commercially available needle-based CGMs (Abbott Freestyle Libre 3 and Dexcom G7) were inserted in the dominant arm to record IF glucose levels every five minutes, while finger-pricking via a Nova Biomedical standard glucometer provided blood glucose levels every 10 minutes. To induce dynamic changes in glucose levels, two 75 mg glucose drinks were administered during the trial.

During the in vivo clinical testing on humans, participant movements over extended periods, variations in skin moisture, and differences in the pressure of the forearm against the device all contribute to signal variability. Additionally, melanin in the skin is subject to photobleaching over time, which can influence the optical signals. To address these complexities, a preliminary data analysis strategy was applied, which included baseline correction and detrending to remove these kinds of slowly varying background signals. Absolute area and sum of slope metrics, as described above, were computed, starting from corrected weighted data, followed by a quadratic calibration of the Raman-based metrics using blood glucose levels as a reference.

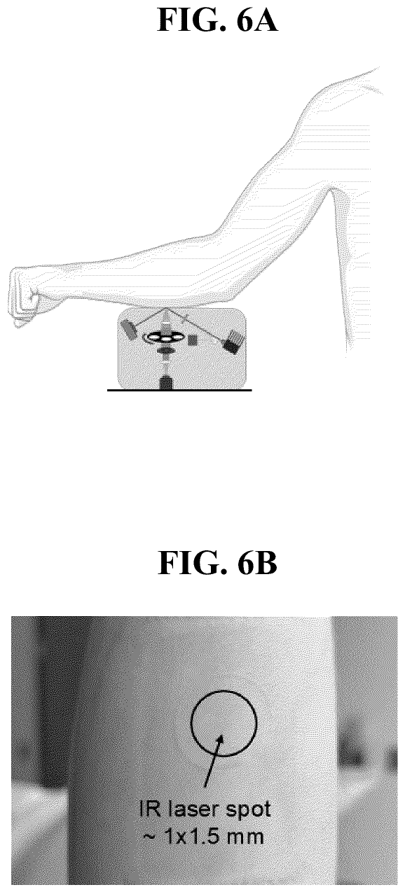

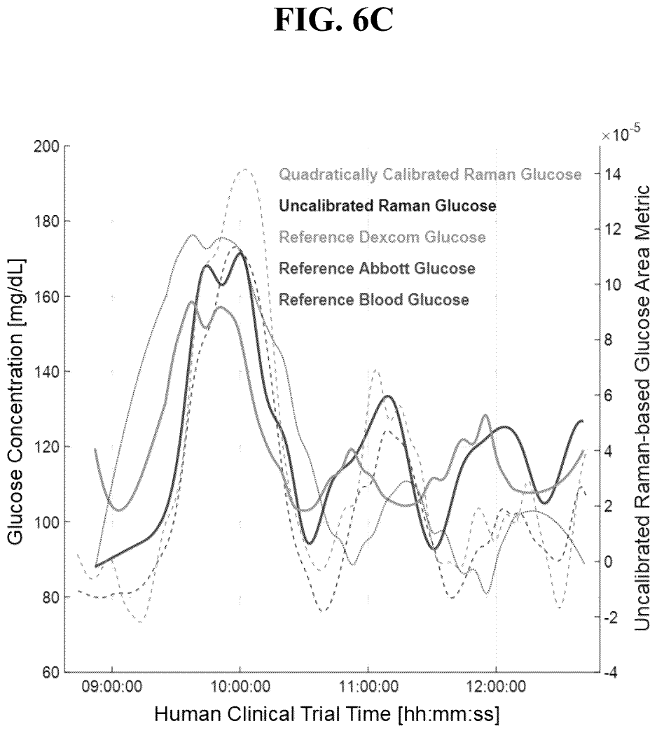

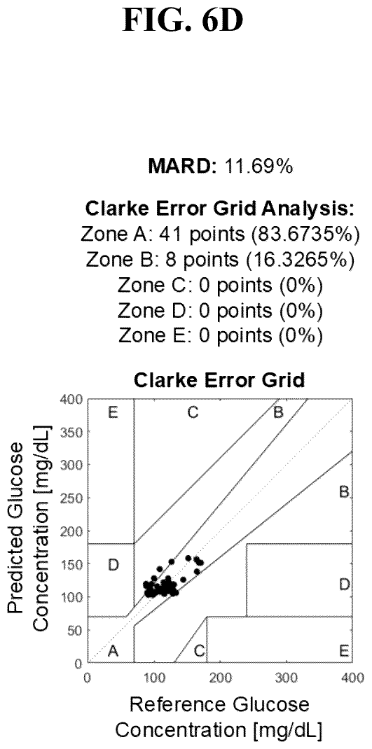

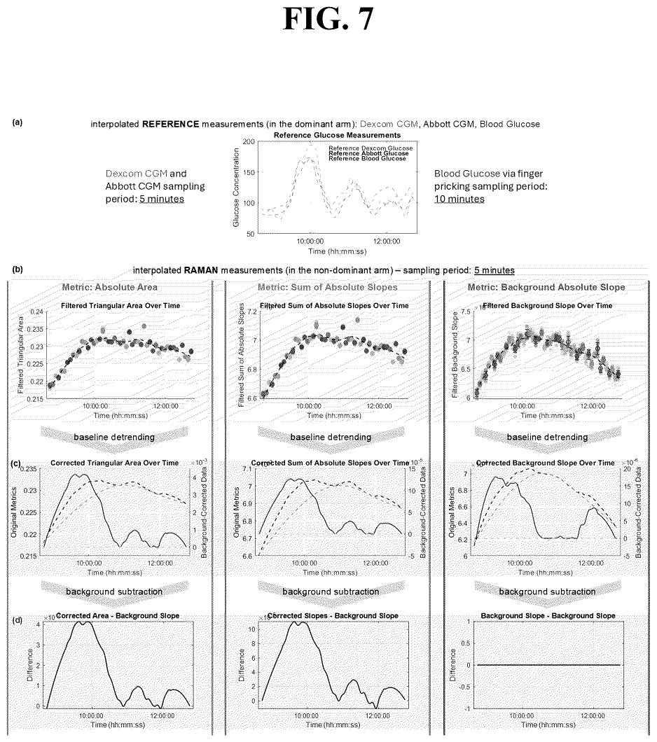

FIGS. 6A-6D illustrate BRS-based in-vivo intra-skin CGM on humans. FIG. 6A shows forearm and portable system configuration for clinical trials on healthy humans. FIG. 6B shows spot of incidence of the 830.35-nm beam onto the participant skin at 120 mW, which indicates that the beam does not produce any hint of skin irritation after the 4-hour-long clinical trial. FIG. 6C shows calibrated glucose levels in blood (mg/dL) via finger pricking (in red), in the IF via Abbott and Dexcom needle-based CGM devices (in dashed shades of grey), and by the BRS CGM device according to the present disclosure (in green). The uncalibrated BRS CGM absolute area metric is plotted referred to the left y-axis (in blue). FIG. 6D shows Clarke error grid analysis of the intra-skin BRS CGM signal compared to the blood glucose levels.

FIG. 7 shows a more detailed explanation of the clinical data processing. Panel (a) of FIG. 7 depicts reference glucose levels measured both in the IF, via two needle-based commercially available sensors (i.e., Abbott Freestyle 3 and Dexcom G7 CGMs) in the upper dominant arm of the participant, sampling every 5 minutes, and directly in blood via finger-pricking through a Nova Biomedical standard glucometer, sampling every 10 minutes. The curves shown are spline interpolations. Panel (b) of FIG. 7 depicts measurement using the BRS portable system to quantify glucose levels in the IF, with a sampling period of 5 minutes. Analogously to the data processing pipeline developed for tissue phantoms, two metrics are calculated: the absolute area under the Raman peak of glucose (first column), and the sum of absolute slopes of the Raman peak of glucose (second column). Additionally, due to the complexity of the scattering background given by living human skin, the slope of the sidebands (excluding the signal at the glucose peak) is calculated to account for background variations (third column).

To mitigate noise and smooth the interpolated Raman-based metric curves over time, a second-order Savitzky-Golay filter with a window size of 67 points is applied. Referring to panel (c) of FIG. 7, most likely due to the photobleaching of skin pigments, a smoothly varying background curve was observed, on top of which the metric oscillations sit. Such background was removed through baseline detrending via the adaptive smoothness partial least squares algorithm employing the following parameters: smoothness parameter=10⁵; order=2; decay coefficient=2; maximum number of iterations=200; convergence threshold=10⁻⁴. Referring to panel (d) of FIG. 7, to compensate for the background variations and limit the impact of background-only dynamics to the Raman peak-related metrics of area and slope, the analysis was performed by subtracting the background metric (third column) from the absolute area metric (first column) and from the sum of absolute slopes metric (second column).

The Raman-based CGM system according to the present disclosure achieved a MARD of 11.69%, which represents excellent accuracy for clinical-level CGM systems. The Clarke error grid analysis further supports the robustness of the system, with 100% of observations falling within zones A and B, indicating no clinically significant errors. Specifically, 83.67% of observations were in zone A, representing clinically accurate results, and 16.33% were in zone B, indicating benign deviations with no impact on therapeutic decisions.

These results demonstrate that the optical Raman-based CGM system according to the present disclosure can provide accurate and safe CGM in real-world conditions.

Unlike conventional Raman spectroscopy systems, the compact, portable and noninvasive BRS system according to the present disclosure enables targeted detection of the glucose Raman signature without the need to collect the full-spectrum signal. Avoiding bulky diffraction gratings and expensive CCD cameras typical of standard Raman spectroscopy, a significant improvement is realized in terms of size, complexity, and cost with respect to other noninvasive CGM devices. The streamlined and efficient system, featuring a total measurement time under one minute, can provide practical CGM in in-vitro and in-vivo applications. The combination of this device with a systematic data processing pipeline, focused on intra-spectrum relative changes rather than absolute intensity, makes the solution an outstandingly robust method across varying experimental conditions. The preliminary clinical data from healthy humans showed promising perspectives. This innovative approach can reflect improvement to the technical field of glucose monitoring and can offer portability, accessibility, accuracy and continuity for both clinical and personal health management applications.

It should be understood that the subject matter defined in the appended claims is not necessarily limited to the specific implementations described above. The specific implementations described above are disclosed as examples only.

### REFERENCES

(1) Guzman-Vilca, W. C.; Carrillo-Larco, R. M. Number of People with Type 2 Diabetes Mellitus in 2035 and 2050: A Modelling Study in 188 Countries. Curr. Diabetes Rev. 2025, 21 (1), e120124225603. https://doi.org/10.2174/0115733998274323231230131843.
(2) Flockhart, M.; Larsen, F. J. Continuous Glucose Monitoring in Endurance Athletes: Interpretation and Relevance of Measurements for Improving Performance and Health. Sports Med. 2024, 54 (2), 247-255. https://doi.org/10.1007/s40279-023-01910-4.
(3) Zou, Y.; Chu, Z.; Guo, J.; Liu, S.; Ma, X.; Guo, J. Minimally Invasive Electrochemical Continuous Glucose Monitoring Sensors: Recent Progress and Perspective. Biosens. Bioelectron. 2023, 225, 115103. https://doi.org/10.1016/j.bios.2023.115103.
(4) Todaro, B.; Begarani, F.; Sartori, F.; Luin, S. Is Raman the Best Strategy towards the Development of Non-Invasive Continuous Glucose Monitoring Devices for Diabetes Management?Front. Chem. 2022, 10, 994272. https://doi.org/10.3389/fchem.2022.994272.
(5) Reddy, P. S. K.; Mahesh, D.; Uday Teja, C.; Janaki, M.; Mannem, K. Non-Invasive Glucose Monitoring Using NIR Spectroscopy. J. Phys. Conf. Ser. 2022, 2325 (1), 012021. https://doi.org/10.1088/1742-6596/2325/1/012021.
(6) Lubinski, T.; Plotka, B.; Janik, S.; Canini, L.; Mantele, W. Evaluation of a Novel Noninvasive Blood Glucose Monitor Based on Mid-Infrared Quantum Cascade Laser Technology and Photothermal Detection. J. Diabetes Sci. Technol. 2021, 15 (1), 6-10. https://doi.org/10.1177/1932296820936634.
(7) Shaikh, F.; Haworth, N.; Wells, R.; Bishop, J.; Chatterjee, S. K.; Banerjee, S.; Laha, S. Compact Instrumentation for Accurate Detection and Measurement of Glucose Concentration Using Photoacoustic Spectroscopy. IEEE Access 2022, 10, 31885-31895. https://doi.org/10.1109/ACCESS.2022.3158945.
(8) Gao, M.; Guo, D.; Wang, J.; Tan, Y.; Liu, K.; Gao, L.; Zhang, Y.; Ding, Z.; Gu, Y.; Li, P. High-Accuracy Noninvasive Continuous Glucose Monitoring Using OCT Angiography-Purified Blood Scattering Signals in Human Skin. Biomed. Opt. Express 2024, 15 (2), 991. https://doi.org/10.1364/BOE.506092.
(9) Purvinis, G.; Cameron, B. D.; Altrogge, D. M. Noninvasive Polarimetric-Based Glucose Monitoring: An in Vivo Study. J. Diabetes Sci. Technol. 2011, 5 (2), 380-387. https://doi.org/10.1177/193229681100500227.
(10) Czarnik, A. W.; James, T. D. Fluorescent Chemosensors in the Creation of a Commercially Available Continuous Glucose Monitor. ACS Sens. 2024, 9 (12), 6320-6326. https://doi.org/10.1021/acssensors.4c02403.
(11) Zhang, M.; Liu, J.; Wang, N.; Zhang, B.; Gao, F.; Wang, M.; Song, Q. High-Precision Sensor for Glucose Solution Using Active Multidimensional Feature THz Spectroscopy. Biomed. Opt. Express 2024, 15 (3), 1418. https://doi.org/10.1364/BOE.515588.
(12) Omer, A. E.; Shaker, G.; Safavi-Naeini, S.; Kokabi, H.; Alquie, G.; Deshours, F.; Shubair, R. M. Low-Cost Portable Microwave Sensor for Non-Invasive Monitoring of Blood Glucose Level: Novel Design Utilizing a Four-Cell CSRR Hexagonal Configuration. Sci. Rep. 2020, 10 (1), 15200. https://doi.org/10.1038/s41598-020-72114-3.
(13) Jang, C.; Lee, H.-J.; Yook, J.-G. Radio-Frequency Biosensors for Real-Time and Continuous Glucose Detection. Sensors 2021, 21 (5), 1843. https://doi.org/10.3390/s21051843.
(14) Meyhofer, S.; Wilms, B.; Ihling, F.; Windjager, A.; Kalscheuer, H.; Augustinov, A.; Herrmann, V.; Lehnert, H.; Schmid, S. M. Evaluation of a Near-infrared Light Ultrasound System as a Non-invasive Blood Glucose Monitoring Device. Diabetes Obes. Metab. 2020, 22 (4), 694-698. https://doi.org/10.1111/dom.13914.
(15) Sanai, F.; Sahid, A. S.; Huvanandana, J.; Spoa, S.; Boyle, L. H.; Hribar, J.; Wang, D. T.-Y.; Kwan, B.; Colagiuri, S.; Cox, S. J.; Telfer, T. J. Evaluation of a Continuous Blood Glucose Monitor: A Novel and Non-Invasive Wearable Using Bioimpedance Technology. J. Diabetes Sci. Technol. 2023, 17 (2), 336-344. https://doi.org/10.1177/19322968211054110.
(16) Lombardi, S.; Bocchi, L.; Francia, P. Photoplethysmography and Artificial Intelligence for Blood Glucose Level Estimation in Diabetic Patients: A Scoping Review. IEEE Access 2024, 12, 178982-178996. https://doi.org/10.1109/ACCESS.2024.3508467.
(17) Kapur, R.; Kumar, Y.; Sharma, R.; Sharma, S.; Singh, E.; Rohilla, D.; Kanwar, V.; Kumar, B.; Bhavsar, A.; Dutt, V. GlucoBreath: An IoT, ML, and Breath-Based Non-Invasive Glucose Meter. IEEE Access 2024, 12, 59346-59360. https://doi.org/10.1109/ACCESS.2024.3392015.
(18) Zafar, H.; Channa, A.; Jeoti, V.; Stojanović, G. M. Comprehensive Review on Wearable Sweat-Glucose Sensors for Continuous Glucose Monitoring. Sensors 2022, 22 (2), 638. https://doi.org/10.3390/s22020638.
(19) Jin, X.; Cai, A.; Xu, T.; Zhang, X. Artificial Intelligence Biosensors for Continuous Glucose Monitoring. Interdiscip. Mater. 2023, 2 (2), 290-307. https://doi.org/10.1002/idm2.12069.
(20) Kang, J. W.; Lue, N.; Kong, C.-R.; Barman, I.; Dingari, N. C.; Goldfless, S. J.; Niles, J. C.; Dasari, R. R.; Feld, M. S. Combined Confocal Raman and Quantitative Phase Microscopy System for Biomedical Diagnosis. Biomed. Opt. Express 2011, 2 (9), 2484. https://doi.org/10.1364/BOE.2.002484.
(21) Kang, J. W.; Park, Y. S.; Chang, H.; Lee, W.; Singh, S. P.; Choi, W.; Galindo, L. H.; Dasari, R. R.; Nam, S. H.; Park, J.; So, P. T. C. Direct Observation of Glucose Fingerprint Using in Vivo Raman Spectroscopy. Sci. Adv. 2020, 6 (4), eaay5206. https://doi.org/10.1126/sciadv.aay5206

What is claimed is:

1. A method for noninvasive continuous glucose monitoring (CGM) using bandpass Raman spectroscopy (BRS), the method comprising:
irradiating, by a light source, light to a subject;
detecting, by a photodetector, the light that is reflected from the subject;
obtaining intensities of peaks at one or more predetermined Raman bands in a Raman spectrum occurring due to Raman shift, wherein the one or more predetermined Raman bands include a glucose Raman peak and two sidebands;
obtaining at least one glucose metric based on the intensities of peaks at the one or more predetermined Raman bands; and
determining a glucose level associated with the subject based on the at least one glucose metric,
wherein the at least one glucose metric corresponds to an area of a triangle, which is defined by the intensities of the glucose Raman peak and the two sidebands, on a signal intensity—Raman shift plot.

2. The method of claim 1, wherein the light comprises infrared (IR) light.

3. The method of claim 2, wherein a wavelength of the IR light is about 830 nm.

4. The method of claim 3, wherein the glucose Raman peak corresponds to a peak at about 1125 cm⁻¹, and
wherein the two sidebands correspond to peaks at about 950 cm⁻¹ and about 1175 cm⁻¹.

5. The method of claim 4, wherein the glucose Raman peak is obtained using a filter having a central wavelength of about 915.5 nm, and the two sidebands are obtained using filters having central wavelengths of about 901.3 nm and about 920.15 nm, respectively.

6. The method of claim 1, further comprising:
normalizing raw signal of the intensities by dividing by correspondent reference signal, thereby obtaining a corrected Raman data,
wherein the reference signal is obtained by routing the light from the light source to a photodetector without directing it to the subject.

7. The method of claim 6, further comprising:
subtracting signals that represent lowest glucose concentration from the corrected Raman data, thereby obtaining a corrected adjusted Raman data.

8. The method of claim 6, further comprising:
applying respective weights to each Raman band by optical bandwidth of corresponding bandpass filter, thereby obtaining a corrected weighted Raman data.

9. The method of claim 8, further comprising:
subtracting signals that represent lowest glucose concentration from the corrected weighted Raman data, thereby obtaining a corrected weighted adjusted Raman data.

10. The method of claim 1, wherein the glucose level associated with the subject is determined based on mapping the at least one glucose metric to a quadratic fitting of prior measurements of plurality of glucose concentrations.

11. A method for noninvasive continuous glucose monitoring (CGM) using bandpass Raman spectroscopy (BRS), the method comprising:
irradiating, by a light source, light to a subject;
detecting, by a photodetector, the light that is reflected from the subject;
obtaining intensities of peaks at one or more predetermined Raman bands in a Raman spectrum occurring due to Raman shift, wherein the one or more predetermined Raman bands include a glucose Raman peak and two sidebands;
obtaining at least one glucose metric based on the intensities of peaks at the one or more predetermined Raman bands; and
determining a glucose level associated with the subject based on the at least one glucose metric,
wherein the at least one glucose metric corresponds to a sum of:
(i) absolute value of a first slope formed by a first line segment connecting the glucose Raman peak and one of the two sidebands on a signal intensity—Raman shift plot; and
(ii) absolute value of a second slope formed by a second line segment connecting the glucose Raman peak and the other of the two sidebands on the signal intensity—Raman shift plot.

12. The method of claim 11, wherein the light comprises infrared (IR) light.

13. The method of claim 12, wherein a wavelength of the IR light is about 830 nm.

14. The method of claim 13, wherein the glucose Raman peak corresponds to a peak at about 1125 cm⁻¹, and
wherein the two sidebands correspond to peaks at about 950 cm⁻¹ and about 1175 cm⁻¹.

15. The method of claim 14, wherein the glucose Raman peak is obtained using a filter having a central wavelength of about 915.5 nm, and the two sidebands are obtained using filters having central wavelengths of about 901.3 nm and about 920.15 nm, respectively.

16. The method of claim 11, further comprising:
normalizing raw signal of the intensities by dividing by correspondent reference signal, thereby obtaining a corrected Raman data,
wherein the reference signal is obtained by routing the light from the light source to a photodetector without directing it to the subject.

17. The method of claim 16, further comprising:
subtracting signals that represent lowest glucose concentration from the corrected Raman data, thereby obtaining a corrected adjusted Raman data.

18. The method of claim 16, further comprising:
applying respective weights to each Raman band by optical bandwidth of corresponding bandpass filter, thereby obtaining a corrected weighted Raman data.

19. The method of claim 18, further comprising:
subtracting signals that represent lowest glucose concentration from the corrected weighted Raman data, thereby obtaining a corrected weighted adjusted Raman data.

20. The method of claim 11, wherein the glucose level associated with the subject is determined based on mapping the at least one glucose metric to a quadratic fitting of prior measurements of plurality of glucose concentrations.
